(19) United States
(12) Patent Application Publication
(10) Pub. No.: US 2026/0115258 A1
(43) Pub. Date: Apr. 30, 2026

(54) PEPTIDE COMPOSITIONS FOR TREATING HAIR LOSS

(71) Applicant: RESTORE BIOLOGICS HOLDINGS, LLC, Orlando, FL (US)

(72) Inventors: Trillitye Paullin, Durham, NC (US); Tracy Yourke, Birmingham, MI (US)

(21) Appl. No.: 19/266,834

(22) Filed: Jul. 11, 2025

Related U.S. Application Data
(60) Provisional application No. 63/670,549, filed on Jul. 12, 2024.

(51) Int. Cl. A61K 38/22; A61K 9/00; A61K 38/10; A61P 17/14

(52) U.S. Cl. CPC A61K 38/2292; A61K 9/0014; A61K 38/10; A61P 17/14

(57) ABSTRACT

Peptide compositions are provided to treat human hair loss, said compositions include a combination of peptides for stimulating hair regrowth. The synergistic effects of this combination target the hair follicles and surrounding cellular structures, promoting tissue regeneration, reducing inflammation, and enhancing cellular repair. This innovative approach offers a more effective and targeted treatment for hair loss, addressing the limitations of existing therapies by focusing on the underlying biological processes involved in hair growth.

### CROSS-REFERENCE TO RELATED APPLICATIONS

This application claims priority to U.S. Provisional Patent Application No. 63/670,549, entitled “PEPTIDE COMPOSITIONS FOR TREATING HAIR LOSS”, filed on Jul. 12, 2024, the contents of which are hereby incorporated herein in their entirety.

### REFERENCE TO A SEQUENCE LISTING

The application contains a Sequence Listing which has been submitted electronically in XML format and is hereby incorporated by reference in its entirety. Said XML copy, created on Jul. 10, 2025, is named Peptide Compositions for Treating Hair Loss—P320045US02.xml and is 2,714 bytes in size.

### TECHNICAL FIELD

The disclosed compositions, processes and methods are directed to treatment of hair loss.

### BACKGROUND

Hair loss affects millions of people worldwide, leading to significant psychological and social impacts. Traditional treatments for hair loss include medications such as minoxidil and finasteride, hair transplantation surgeries, and the use of various topical solutions and shampoos. However, these treatments often have limited efficacy, potential side effects, and high costs. Additionally, many of these approaches only address hair loss symptoms rather than targeting the underlying biological processes involved in hair regrowth. Recent advancements in biotechnology have highlighted the potential of peptides as therapeutic agents due to their ability to modulate cellular functions and promote tissue regeneration. However, there has been limited exploration into how the synergistic potential in peptide combinations might be leveraged to develop more effective treatments for hair loss.

### BRIEF DESCRIPTION OF THE DRAWINGS

FIG. 1 is a graph showing results of an MTT assay evaluating the effects of multiple ratios of three peptides on cell proliferation.

FIG. 2 shows images from a wound healing assay comparing a control to a treatment with a peptide combination in accordance with one embodiment.

FIG. 3 shows images comparing the effect of treatment with a peptide combination in accordance with one embodiment on cellular migration in human hair outer root sheath cells.

### TERMS AND DEFINITIONS

As used herein, the terms “peptide” and “polypeptide” are used interchangeably to designate a series of amino acid residues, connected to each other by peptide bonds between the alpha-amino and carboxy groups of adjacent residues. These may include modified amino acids (e.g., phosphorylated, glycated, glycosylated, etc.) and amino acid analogs. “Polypeptide” is often used in reference to relatively large polypeptides, whereas the term “peptide” is often used in reference to small polypeptides, but usage of these terms in the art overlaps.

The identities of amino acids in a polypeptide are based on the structure of the functional group (R group) on the polypeptide backbone at a given position. Naturally occurring amino acid identities are (name/3-letter code/one-letter code): alanine/ala/A; arginine/arg/R; asparagine/asn/N; aspartic acid/asp/D; cysteine/cys/C; glutamine/gln/Q; glutamic acid/glu/E; glycine/gly/G; histidine/his/H; isoleucine/ile/I; leucine/leu/L; lysine/lys/K; methionine/met/M; phenylalanine/phe/F; proline/pro/P; serine/ser/S; threonine/thr/T; tryptophan/trp/W; tyrosine/tyr/Y; and valine/val/V.

“Variant,” as used herein refers to a polypeptide, sequence, or molecule that is substantially homologous to a naturally occurring or reference member, but which is different from that of the native or reference member because of one or a plurality of deletions, insertions, substitutions, modifications, etc. Variant amino acid or nucleic acid sequences can be at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and variant sequence can be determined, for example, by comparing the two sequences using freely available computer programs commonly employed for this purpose on the world wide web (e.g., BLASTp with default settings).

An amino acid within a polypeptide molecule may be substituted to create a variant of that polypeptide. The amino acid residue can be replaced by a residue having similar physiochemical characteristics, that is a ‘conservative substitution’—e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, for example based on size, charge, polarity, hydrophobicity, chain rigidity/orientation, etc., are well known in the art of protein engineering. Polypeptides comprising conservative amino acid substitutions can be tested in any one of the assays described herein to confirm that a desired activity, e.g. binding, specificity, and/or function of a native or reference polypeptide is achieved.

The terms “effective amount” or “therapeutically effective amount” mean an amount of a drug, composition, compound, treatment, or therapy of the present disclosure that alone, or in combination with other therapies, (i) treats the particular disease, condition, or disorder, (ii) attenuates, ameliorates, or eliminates one or more symptoms of the particular disease, condition, or disorder, or (iii) prevents or delays, the onset of one or more symptoms of the particular disease, condition, or disorder described herein. The term can encompass an amount that improves overall therapy, reduces, or avoids symptoms or causes of disease, or enhances the therapeutic efficacy or synergizes with another therapeutic agent.

The terms “treat,” “treating,” and “treatment” refer to eliminating, reducing, suppressing, or ameliorating, either temporarily or permanently, either partially or completely, a clinical symptom, manifestation or progression of an event, disease or condition associated with immune disorders and diseases described herein. As is recognized in the pertinent field, methods and compositions employed as therapies may reduce the severity of a given disease state but need not abolish every manifestation of the disease to be regarded as useful. Similarly, a prophylactically administered treatment need not be completely effective in preventing the onset of a condition to constitute a viable prophylactic method or agent. Simply reducing the impact of a disease (for example, as disclosed herein, hair loss, etc. and/or reducing the number or severity of associated symptoms, or by increasing the effectiveness of another treatment, or by producing another beneficial effect), or reducing the likelihood that the disease will occur or worsen in a subject, is sufficient.

### DETAILED DESCRIPTION

The present disclosure describes compositions comprising novel peptide combinations designed to treat human hair loss by stimulating hair regrowth. In particular, the present disclosure is concerned with peptides that individually have shown promise in various regenerative medicine applications, but for which there has been limited exploration into their combined effects on hair follicle stimulation and hair regrowth. The compositions described herein seek to leverage the synergistic potential of these peptides to create a novel treatment for hair loss. By combining these peptides in specific concentrations, the goal is to develop a more effective and targeted approach to promoting hair regrowth. This innovative method aims to overcome the limitations of existing treatments by addressing the root causes of hair loss at the cellular level, offering a potentially safer and more efficient solution for individuals suffering from hair thinning and balding.

Compositions according to the present disclosure can comprise a combination of peptides, each of which is effective in promoting responses associated with hair growth. For example, the peptide combination may be effective in initiating and/or extending the active growth phase of hair follicles. The peptides may be present in a combination and in respective amounts that are selected to provide enhanced effectiveness in promoting hair growth. To that end, each peptide may be selected for its effect on one or more processes directly or indirectly involved in hair growth. In some embodiments, the combination may include at least one peptide having regenerative properties, particularly the ability to stimulate tissue regeneration, as well as the ability to promote wound healing and anti-inflammatory responses. In some embodiments, the combination may include at least one peptide that promotes cell migration, reduces inflammation, and enhances tissue repair, particularly in hair follicle cells.

Peptides contemplated for combination in the compositions of the present disclosure include, but are not limited to: a complex of the tripeptide glycyl-L-histidyl-L-lysine with a copper ion (GHK-Cu); thymic peptide thymosin beta-4, and the human body protection compound-derived pentadecapeptide BPC-157. In various embodiments, a composition can include an effective amount of a peptide combination comprising at least two of these peptides or variants thereof. In certain embodiments, the peptide combination comprises GHK-Cu, thymosin beta-4, and BPC-157 or variants thereof. In some embodiments, the composition includes a variant of thymosin beta-4 having a sequence that is at least 80%, at least 85%, at least 90%, at least 91%, at least 92%, at least 93%, at least 94%, at least 95%, at least 96%, at least 97%, at least 98% or at least 99% identity to the sequence Ser Asp Lys Pro Asp Met Ala Glu Ile Glu Lys Phe Asp Lys Ser Lys Leu Lys Lys Thr Glu Thr Gln Glu Lys Asn Pro Leu Pro Ser Lys Glu Thr Ile Glu Gln Glu Lys Gln Ala Gly Glu Ser (SEQ ID NO: 1). In some embodiments, the composition includes a variant of BPC-157 having a sequence that is at least 80%, at least 85%, at least 90%, at least 91%, at least 92%, at least 93%, at least 94%, at least 95%, at least 96%, at least 97%, at least 98% or at least 99% identity to the sequence Gly Glu Pro Pro Pro Gly Lys Pro Ala Asp Asp Ala Gly Leu Val (SEQ ID NO: 2).

In some embodiments, an amount of the peptide combination present in the composition can be about 0.01 wt % to about 5 wt %, or more particularly the amount can be about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.2 wt %, about 1.4 wt %, about 1.6 wt %, about 1.8 wt %, about 2.0 wt %, about 2.2 wt %, about 2.4 wt %, about 2.6 wt %, about 2.8 wt %, about 3.0 wt %, about 3.2 wt %, about 3.4 wt %, about 3.6 wt %, about 3.8 wt %, about 4.0 wt %, about 4.2 wt %, about 4.4 wt %, about 4.6 wt %, about 4.8 wt %, and about 5.0 wt %. Within this total amount, each of the peptides can be present in the composition in specific concentrations selected to maximize their synergistic effects on hair regrowth. In some embodiments, each of the constituent peptides can be present in the composition in approximately equal amounts. In a particular embodiment, the composition comprises about 0.8 wt % to about 1.2 wt % each of GHK-Cu, thymosin beta-4 and BPC-157. In some embodiments, GHK-Cu and BPC-157 can each be present at a ratio of about 1:1 to about 3:1 with respect to thymosin beta-4. In some embodiments, a composition can include GHK-Cu, thymosin beta-4 and BPC-157, where GHK-Cu and BPC-157 are present at about 0.02 wt % to about 2 wt % and thymosin beta-4 is present at about 0.01 wt % to about 1 wt %.

The present disclosure also includes compositions the provide a more concentrated formulation of the peptide combination. In some embodiments, an amount of the peptide combination present in such compositions can be about 5 wt % to about 60 wt %, or more particularly about 10 wt % to about 50 wt %. In particular embodiments the amount is about 5 wt %, about 10 wt %, about 15 wt %, about 20 wt %, about 25 wt %, about 30 wt %, about 35 wt %, about 40 wt %, about 45 wt %, about 50 wt %, about 55 wt %, or about 60 wt %. Within this total amout, each of the peptides can be present in the composition in specific concentrations selected to maximize their synergistic effects on hair regrowth. In some embodiments, GHK-Cu and BPC-157 can each be present at a ratio of about 1:1 to about 3:1 with respect to thymosin beta-4. In particular embodiments GHK-Cu and BPC-157 are present in a ratio of about 2:1 with respect to thymosin beta-4, such that the GHK-Cu:thymosin beta-4:BPC-157 ratio is about 2:1:2 (which may be expressed as a multiple thereof, such as 10:5:10). In some embodiments, the composition can include GHK-Cu, thymosin beta-4 and BPC-157, where GHK-Cu and BPC-157 are present at about 2 wt % to about 20 wt % and thymosin beta-4 is present at about 1 wt % to about 10 wt %.

A method of making a peptide composition for use in treating hair loss can comprise synthesizing one or more of a combination of peptides. In some embodiments, the peptides are manufactured using standard solution phase methodology. Suitable synthesis methodologies include solid phase peptide synthesis, liquid phase peptide synthesis, and microwave-assisted peptide synthesis. In an exemplary method, the peptides are generated using a solid phase peptide synthesis (SPPS) method. In some embodiments, the peptide is then purified. In some embodiments, the peptide is purified by HPLC purification. In the case of a peptide-metal complex, synthesis can further involve adding the appropriate metal ion to the synthesized peptide. For example, GHK-Cu can be produced by mixing equimolar solutions of GHK and a copper salt such as CuCl₂ or copper acetate.

In some embodiments, a peptide composition can comprise a peptide combination in which two or more linked peptides are present as a complex. In particular embodiments, each complex includes at least two different peptides linked by a crosslinking agent. In some embodiments a crosslinking agent may be selected that is generally reactive with peptides, particularly with amine groups. In some embodiments, a crosslinking agent may be used that is more selective for particular peptides, for example through reactivity with particular side chains. In certain embodiments, the crosslinking agent may be polyfunctional, and more particularly bifunctional or trifunctional. In various embodiments, crosslinking agents in accordance with various embodiments include, without limitation, glutaraldehyde, disuccinimidyl suberate (DSS), bis(sulfosuccinimidyl) suberate (BS3), ethylene glycol bis(succinimidyl succinate) (EGS), N-hydroxysuccinimide (NHS) esters, maleimides, and polyethylene glycol (PEG).

It will be understood that crosslinking agents may be selected to create complexes from different combinations of peptides. In some embodiments, a bifunctional crosslinking agent links the N-terminus of a first peptide with a lysine residue of a second peptide. In an embodiment, a peptide combination comprises a complex of GHK-Cu and thymosin beta-4 or a variant thereof, where a bifunctional crosslinking agent links the N-terminus of GHK-Cu with a lysine residue of the thymosin beta-4 or its variant. In another embodiment, a peptide combination comprises a complex of thymosin beta-4 or a variant thereof and BPC-157 or a variant thereof, where a bifunctional crosslinking agent links the N-terminus of the thymosin beta-4 or its variant with a lysine residue of the BPC-157 or its variant. In another embodiment, a peptide combination comprises a complex of GHK-Cu, thymosin beta-4 or a variant thereof and BPC-157 or a variant thereof, where a first bifunctional crosslinking agent links the N-terminus of GHK-Cu with a first lysine residue of the thymosin beta-4 or its variant, and a second bifunctional crosslinking agent links a second lysine of thymosin beta-4 to the C-terminus of the BPC-157. It will be appreciated that other crosslinking structures and arrangements may be used to create other complexes.

The peptide combination can be dissolved in a suitable solvent to produce a peptide solution for use in treating hair loss. In some embodiments, a peptide solution can comprise an amount of a peptide combination as discussed above in a solvent, where the peptide combination comprises at least two of GHK-Cu, thymosin beta-4, BPC-157 and variants thereof. Any solvent that is suitable to create a stable and effective solution for topical application may be used, including, without limitation, water, phosphate-buffered saline (PBS), ethanol, propylene glycol, or glycerin.

A peptide composition according to the present disclosure may be provided in powder form for e.g., purposes of storage or for incorporation into solid formulations for treating hair loss. In some embodiments, a powder can be produced from a peptide solution as described above by removing solvent from the solution, such as by spray-drying, freeze drying or lyophilization. The resulting powder can comprise less than about 5 wt %, less than about 2 wt %, less than about 1 wt %, less than about 0.5 wt %, or less than about 0.1 wt % of a liquid component such as water.

In various embodiments, a peptide composition as described above can be incorporated into various formulations suitable as a vehicle for use by topical application. In particular, such formulations can be designed for care of the hair and/or scalp. Such compositions include, without limitation, shampoos, ointments, creams, foams, lotions, conditioners, masks, serums, oils, and dry shampoos. Each composition may be additionally formulated to promote stability and efficacy of the peptides while providing a user-friendly application. In some embodiments, a hair care formulation can be made by combining a peptide composition with a base formulation comprising ingredients selected for a particular hair care application.

One hair care formulation in accordance with the present disclosure is a shampoo. “Shampoo” as used herein refers particularly to cleansing formulations that are applied to wet hair, lathered, and then rinsed away with added water. In some embodiments, a shampoo can comprise about 0.01 wt % to about 5 wt % of a peptide combination as described above. In particular embodiments, the peptide combination comprises GHK-Cu, thymosin beta-4 and BPC-157. In particular embodiments, the shampoo can further comprise one or more ingredients selected from surfactants, conditioning agents, thickeners, fragrances, and preservatives.

Another hair care formulation in accordance with the present disclosure is a rinse-off conditioner. “Rinse-off conditioner” as used herein refers to formulations that are typically applied to wet hair, particularly after shampooing, and then rinsed off with added water. The conditioner is formulated to render the hair more manageable before and after the hair is dried. In some embodiments, a rinse-off conditioner can comprise about 0.01 wt % to about 5 wt % of a peptide combination as described above. In particular embodiments, the peptide combination comprises GHK-Cu, thymosin beta-4 and BPC-157. In particular embodiments, the conditioner can further comprise one or more ingredients selected from emollients, emulsifiers, fragrances and preservatives.

Another hair care formulation in accordance with the present disclosure is a conditioning mask. “Conditioning mask” as used herein refers to conditioning formulations that are applied to all or part of a subject's hair and then left in place for a period of time before being removed, such as by rinsing. In some embodiments, a conditioning mask can comprise about 0.01 wt % to about 5 wt % of a peptide combination as described above. In particular embodiments, the peptide combination comprises GHK-Cu, thymosin beta-4 and BPC-157. In particular embodiments, the conditioning mask can further comprise one or more ingredients selected from conditioning agents, emulsifiers, thickeners, fragrances and preservatives.

Another hair care formulation in accordance with the present disclosure is a leave-in conditioner. “Leave-in conditioner” as used herein refers to conditioning formulations that can be applied to wet or dry hair and left in place for an indefinite period of time. In some embodiments, a leave-in conditioner can comprise about 0.01 wt % to about 5 wt % of a peptide combination as described above. In particular embodiments, the peptide combination comprises GHK-Cu, thymosin beta-4 and BPC-157. In particular embodiments, the leave-in conditioner can further comprise one or more ingredients selected from conditioning agents, humectants, and preservatives.

Another hair care formulation in accordance with the present disclosure is a hair oil. “Hair oil” as used herein refers to formulations that are applied directly to hair, typically to deliver an active ingredient to the hair strands. In some embodiments, a hair oil can comprise about 0.01 wt % to about 5 wt % GHK-Cu, thymosin beta-4 and BPC-157. In particular embodiments, the hair oil can further comprise one or more ingredients selected from carrier oils, essential oils and preservatives.

Another hair care formulation in accordance with the present disclosure is a scalp serum. “Scalp serum” as used herein refers to formulations directed particularly to treating the scalp. Scalp serums are typically applied directly to—and optionally massaged into—the scalp. In some embodiments, a scalp serum can comprise about 0.01 wt % to about 5 wt % of a peptide combination as described above. In particular embodiments, the peptide combination comprises GHK-Cu, thymosin beta-4 and BPC-157. In particular embodiments, the scalp serum can further comprise one or more ingredients selected from humectants and preservatives.

Another hair care formulation in accordance with the present disclosure is a dry shampoo. The term “dry shampoo” as used herein refers to a product that is effective to cleanse hair without the need for rinsing with water following application. More specifically, a dry shampoo can be applied to hair and then optionally be distributed through the hair e.g. by brushing, combing or massaging. In some embodiments, a dry shampoo can comprise about 0.01 wt % to about 5 wt % of a peptide combination as described above. In particular embodiments, the peptide combination comprises GHK-Cu, thymosin beta-4 and BPC-157. In particular embodiments, the dry shampoo can further comprise one or more ingredients selected from absorbent powders, conditioning agents, preservatives, fragrances, colorants and propellants. Suitable absorbent powders include, but are not limited to, rice starch, kaolin clay, cornstarch, potato starch, tapioca starch, wheat starch, cassaya starch and combinations thereof.

Surfactants used in the above formulations where applicable may be selected from, but are not limited to, anionic surfactants such as sodium lauryl sulfate, ammonium laureth sulfate, sodium laureth sulfate, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth sulfate, sodium tridecyl sulfate, sodium methyl lauroyl taurate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, sodium dodecyl benzene sulfonate; amphoteric surfactants such as cocobetaine, cocamidopropyl betaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine; and nonionic surfactants such as cocamide MEA, cocamide DEA, cocamidopropylamine oxide, lauryl dimethylamine oxide, cocodimethylamine oxide and laurylamidopropyl amine oxide.

Conditioning agents used in the above formulations where applicable can include, but are not limited to, shea butter, panthenol, silicone quaterniums and other quaternary ammonium compounds and amidoamines. Suitable thickeners include, but are not limited to, xanthan gum, guar gum, hydroxyethyl cellulose, methyl cellulose, hydroxymethyl cellulose, starch and starch derivatives.

Emollients used in the above formulations where applicable can include, but are not limited to, glycerin, coconut oil, mineral oil, isopropyl myristate, propylene glycol, cetyl alcohol, and lanolin.

Humectants used in the above formulations where applicable can include, but are not limited to, glycerin, propylene glycol, butylene glycol, pentylene glycol, hexylene glycol, dipropylene glycol, diethylene glycol, diglycerol, hyaluronic acid, sorbitol, xylitol, inulin, hyaluronic acid or mixtures thereof.

Emulsifiers used in the above formulations where applicable include but are not limited to fatty alcohols such as decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol and mixtures thereof, cetrimonium chloride, glucosides, emulsifying esters and ester oils.

Carrier oils used in the above formulations where applicable include, but are not limited to, argan oil, jojoba oil, coconut oil and olive oil.

Essential oils used in the above formulations where applicable include, but are not limited to, lemongrass oil, helichrysum oil, lavender oil, clary sage oil, cedarwood oil, eucalyptus oil, thyme oil, bergamot oil, tea tree oil, rosemary oil, and peppermint oil.

Absorbent powders used in the above formulations where applicable include, but are not limited to, rice starch, kaolin clay, cornstarch, potato starch, tapioca starch, wheat starch, cassaya starch and combinations thereof.

A method of treating hair loss can comprise applying a peptide combination to an area of skin affected by hair loss, e.g., the scalp, in such a way as to bring the peptide combination into contact with hair follicles. In various embodiments, this can involve applying a hair care formulation comprising the peptide combination to the scalp and/or hair. In many cases, the hair care formulation can be designed to impart a beneficial effect to the scalp and/or hair (e.g., cleansing, conditioning, coloring) in addition to delivering the peptide combination. Accordingly, the treatment regimen and mode of application may be determined by how the hair care formulation is used to impart the other benefits of the formulation. In each case, the formulation can be designed so that the manner and frequency of use for products of its kind will also deliver an effective amount of the peptide combination to hair follicles so as to remedy hair loss. For example, shampoos and rinse-off conditioners are typically applied during regular hair washing routines. Leave-in conditioners, conditioning masks, scalp serums, hair oils and dry shampoos can be used daily or as directed for the desired level of effect to which each is directed. Accordingly, in some embodiments, treating hair loss with a peptide combination delivered by such formulations can comprise applying the formulation daily or as otherwise directed to achieve an effect to which the formulation is directed.

As discussed above, some hair care formulations are designed for use with damp or wet hair, and some further are subsequently removed from the hair, such as by rinsing with water. In some embodiments, treating hair loss using a shampoo, rinse-off conditioner, leave-in conditioner or conditioning mask comprising the peptide combination can comprise wetting an area of skin affected by hair loss and then applying an amount of the formulation to the area. With a shampoo, rinse-off conditioner, or conditioning mask the method can further comprise rinsing the formulation off of the area after a period of time. With some hair care formulations, wetting before application is not needed. Accordingly, in some embodiments, use of a formulation such as a scalp serum, hair oil or dry shampoo can comprise applying the formulation to an area of skin affected by hair loss without the need to wet the area beforehand.

### EXAMPLES

### Example 1: Hair Regrowth Shampoo

A hair regrowth shampoo is formulated from the following ingredients:
Water
Sodium lauryl sulfate
Panthenol
Xanthan gum
Preservatives
GHK-Cu (0.1 wt %)
thymosin beta-4 (0.05 wt %)
BPC-157 (0.1 wt %)

### Procedure:

1. The GHK-Cu, thymosin beta-4, and BPC-157 are dissolved in a small amount of water.
2. Water, sodium lauryl sulfate, panthenol, xanthan gum, and preservatives are combined and mixed to create a shampoo base.
3. The peptide solution is added to the shampoo base and mixed thoroughly.

### Example 2: Daily Hydrating Conditioner

A hydrating conditioner for daily use is formulated from the following ingredients:
Water
Glycerin
Cetearyl alcohol
Preservatives
GHK-Cu (0.2 wt %)
thymosin beta-4 (0.1 wt %)
BPC-157 (0.2 wt %)

### Procedure:

1. The GHK-Cu, thymosin beta-4, and BPC-157 are dissolved in a small amount of water.
2. Water, glycerin, cetearyl alcohol, and preservatives are combined and mixed to create a conditioner base.
3. The peptide solution is added to the conditioner base and mixed thoroughly.

### Example 3: Conditioning Mask

A conditioning mask is formulated from the following ingredients:
Water
Shea butter
Emulsifiers
Thickeners
Preservatives
GHK-Cu (0.1 wt %)
thymosin beta-4 (0.05 wt %)
BPC-157 (0.1 wt %)

### Procedure:

1. The GHK-Cu, thymosin beta-4, and BPC-157 are dissolved in a small amount of water.
2. Water, shea butter, emulsifiers, thickeners, and preservatives are combined and mixed to create a mask base.
3. The peptide solution is added to the mask base and mixed thoroughly.

### Example 4: Leave-In Conditioner

A leave-in conditioner is formulated from the following ingredients:
Water
Silicone quaterniums
Propylene glycol
Preservatives
GHK-Cu (0.1 wt %)
Thymosin beta-4 (0.05 wt %)
BPC-157 (0.1 wt %)

### Procedure:

1. The GHK-Cu, thymosin beta-4, and BPC-157 are dissolved in a small amount of water.
2. Water, silicone quaterniums, propylene glycol, and preservatives are combined and mixed to create a leave-in conditioner base.
3. The peptide solution is added to the leave-in conditioner base and mixed thoroughly.

### Example 5: Scalp Serum

A scalp serum is formulated from the following ingredients:
Water
Hyaluronic acid
Preservatives
GHK-Cu (0.1 wt %)
thymosin beta-4 (0.05 wt %)
BPC-157 (0.1 wt %)

### Procedure:

1. The GHK-Cu, thymosin beta-4, and BPC-157 are dissolved in a small amount of water.
2. Water, hyaluronic acid, and preservatives are combined and mixed to create a serum base.
3. The peptide solution is added to the serum base and mixed thoroughly.

### Example 6: Hair Oil

A hair oil is formulated from the following ingredients:
Argan oil
Jojoba oil
Preservatives
GHK-Cu (0.05 wt %)
thymosin beta-4 (0.02 wt %)
BPC-157 (0.05 wt %)

### Procedure:

1. The GHK-Cu, thymosin beta-4, and BPC-157 are dissolved in a small amount of ethanol as needed.
2. The argan oil, jojoba oil, and preservatives are combined and mixed to create an oil base.
3. The peptide solution is added to the oil base and mixed thoroughly.

### Example 7: Dry Shampoo

A dry shampoo is formulated from the following ingredients:
Rice starch
Kaolin clay
Conditioning agents
Preservatives
GHK-Cu (0.05 wt %)
thymosin beta-4 (0.02 wt %)
BPC-157 (0.05 wt %)

### Procedure:

1. The GHK-Cu, thymosin beta-4, and BPC-157 are dissolved in a small amount of water or ethanol.
2. The rice starch, kaolin clay, conditioning agents, and preservatives are combined and mixed to create a dry shampoo base.
3. The peptide solution is added to the dry shampoo base and mixed thoroughly.

### Example 8: Synthesis of Component Peptides for a Three-Peptide Formulation

Individual component peptides were synthesized using a Biotage® Alstra™ synthesizer, followed by metal chelation where applicable. The synthesis parameters for each were as follows:

### GHK Peptide Synthesis:

Resin: Rink Amide ChemMatrix
Synthesis Scale: 30 mL vial
Sequence: Gly-His-Lys
Coupling Chemistry: Fmoc-based solid-phase peptide synthesis (SPPS)
Final Crude Peptide Mass: 7.564 g (total from multiple synthesis runs)

### Thymosin Beta-4 (Thymulin) Peptide Synthesis:

Resin: Rink Amide ChemMatrix
Synthesis Scale: 30 mL vial
Sequence: Arg-Lys-Asp-Val-Tyr-Gln-Val-Val-Gly-Cys-Thr-Gly-Pro-Glu-Gly-Ser-Leu.
Coupling Chemistry: Fmoc-based SPPS
Final Crude Peptide Mass: 5.432 g (total from multiple synthesis runs)

### BPC-157 Synthesis:

Resin: Rink Amide ChemMatrix
Synthesis Scale: 30 mL vial
Sequence: Gly-Glu-Pro-Pro-Pro-Gly-Lys-Pro-Ala-Asp-Asp-Ala-Gly-Leu-Val
Coupling Chemistry: Fmoc-based SPPS
Final Crude Peptide Mass: 7.754 g (total from multiple synthesis runs)

### Metal Complexation Processing:

### GHK-Cu Complexation (Copper Tripeptide-1)

Goal: Chelate Copper (II) Acetate Monohydrate with the synthesized GHK peptide to form GHK-Cu. All calculations were scaled up to the total peptide mass.
1. Dissolution:
   1 g of GHK Peptide dissolved in 294 mL of 10 mM Ammonium Acetate buffer (pH 7.4).
2. Copper Acetate Addition:
   530 mg of Copper (II) Acetate Monohydrate was dissolved in a small portion of buffer.
   Slowly added to the GHK solution while stirring.
3. Reaction & Mixing:
   Stirred at room temperature (˜22° C.) for 1-2 hours.
   Solution turned light blue upon complexation.
4. Final Solution Volume: 2000 mL of GHK-Cu solution.

### TB-Zinc Complexation (Zinc Thymulin)

Goal: Chelate Zinc Acetate Dihydrate with the synthesized Thymulin peptide to form TB-Zinc. All calculations were scaled up to the total peptide mass.
1. Dissolution:
   1 g of Thymulin Peptide dissolved in 55 mL of 10 mM Tris-HCl buffer (pH 7.4).
2. Zinc Acetate Addition:
   120 mg of Zinc Acetate Dihydrate was dissolved in a small portion of buffer.
   Slowly added to the Thymulin solution while stirring.
3. Reaction & Mixing:
   Stirred at room temperature (˜22° C.) for 1-2 hours.
   Solution remained clear/slightly opaque after complexation.
4. Final Solution Volume: 200 mL of TB-Zinc solution.

### Example 9: Preparing the Three-Peptide Formulation

### Step 1: Prepare Individual Peptide Solutions

GHK-Cu Solution (No Dilution Needed):
Stock concentration: 3.782 mg/mL
Measure: 52.9 mL

Thymosin Beta-4 (TB-Zinc) Solution (27.16 mg/mL diluted to 1 mg/mL, 100 mL Final Volume):
1. Calculate total dilution needed:
   Stock concentration: 27.16 mg/mL
   Target concentration: 1 mg/ml
   Dilution Factor: 27.16×
1. For 100 mL of final diluted TB-Zinc solution:
   Take 3.68 mL of TB-Zinc stock
   Add 96.32 mL of sterile 10 mM Tris-HCl (pH 7.4) or Milli-Q® Water
   Final volume=100 mL at 1 mg/mL

BPC-157 Solution (Diluted in 1000 mL Milli-Q® Water):
Stock concentration: 7.755 mg/mL
Measure: 25.8 mL

### Step 2: Prepare Final 500 mL Peptide Solution

1. Sterile Mixing
   A 500 mL sterile mixing bottle was used.
   A magnetic stir bar ensured uniform blending.
1. Peptide solutions added to sterile mixing container in this order:
   GHK-Cu Solution: 52.9 mL
   TB-Zinc Solution (Diluted 1 mg/mL): 100 mL
   BPC-157 Solution (7.755 mg/mL): 25.8 mL
1. Add Sterile 10 mM Tris-HCl (pH 7.4) or Milli-Q® Water to bring total volume to 500 mL (approx. 321.3 mL needed).
2. Stir at low speed for 5-10 minutes.

The peptide blend formulation provides a 10:5:10 ratio of GHK-Cu, TB-Zinc, and BPC-157, at a 1 mg/mL total peptide concentration.

### Example 10: Synergistic Effects of GHK-Cu—Thymosin Beta-4—BPC-157 Peptide Formulations for Hair Growth

### Methods

### Cell Lines and Culture Conditions

Human hair outer root sheath cells (HHORSC), hair follicle dermal papilla cells (HFDPC), fibroblasts, and human epidermal keratinocytes (HEK) were selected for this study based on their integral roles in hair follicle health. Each cell type was cultured in standard growth media supplemented with 10% fetal bovine serum (FBS) and 1% penicillin-streptomycin in a humidified incubator at 37° C. with 5% CO₂.

### Peptide Treatment

A formulation of GHK-Cu, Thymosin Beta-4, and BPC-157 was prepared in varying ratios to determine the most effective combination for promoting hair follicle health. Peptide solutions were diluted in sterile phosphate-buffered saline (PBS) and applied to cell cultures at defined concentrations. Control groups were treated with PBS alone.

### Wound Healing Assay

Confluent monolayers of HHORSC and HFDPC cells were subjected to a scratch wound assay to evaluate cellular migration. A standardized scratch was created in the cell monolayers using a sterile pipette tip, and debris was removed by washing with PBS. Cells were then treated with the peptide solutions and incubated for 36 hours. Images were captured at 0, 24, and 36 hours using brightfield microscopy on an Echo Rebel™ Microscope.

### Cell Proliferation Assay

Proliferation of HHORSC and HFDPC cells was assessed using the MTT assay. Cells were seeded in 96-well plates at a density of 10,000 cells per well in 100 μL of culture medium and incubated overnight at 37° C. with 5% CO₂ to allow for attachment. Following incubation, cells were treated with peptide solutions for 48 hours.

After the treatment period, 10 μL of MTT solution (5 mg/mL in PBS) was added to each well, and the plates were incubated for 3 hours at 37° C., allowing viable cells to reduce the MTT to formazan crystals. The medium was then removed, and 100 μL of dimethyl sulfoxide (DMSO) was added to each well to solubilize the formazan crystals. The plates were gently shaken to ensure complete dissolution.

Absorbance was measured at 570 nm using a Promega GloMax® 96-well microplate reader, and relative cell proliferation was determined by normalizing absorbance values to the untreated control group.

### Spheroid Formation Assay

Outer root sheath cells were seeded into ultra-low attachment 96-well plates to form spheroids. Cells were treated with peptide combination, and spheroid morphology, size, and integrity were evaluated using using brightfield microscopy on an Echo Rebel™ Microscope. Parameters analyzed included:
Tightness of spheroids, indicative of cell-cell adhesion.
Spheroid size, reflecting proliferation rates and differentiation states.
Viability, inferred from structural integrity.

### Statistical Analysis

All experiments were performed in triplicate, and data are presented as mean±standard deviation. Statistical significance was assessed using one-way ANOVA followed by Tukey's post-hoc test, with a threshold of p<0.05 unless otherwise noted.

### Proteomic and Pathway Analysis

Proteomic analysis was conducted on HEK, HHORSC, and HFDPC cells treated with the peptide formulation. Proteins were extracted, quantified, and analyzed using ELISA. Data were processed using MaxQuant software and normalized to non-treated control cells. Key pathways analyzed included inflammation modulation, oxidative stress response, ECM remodeling, and follicular proliferation.

### Results

### Cell Proliferation Assay

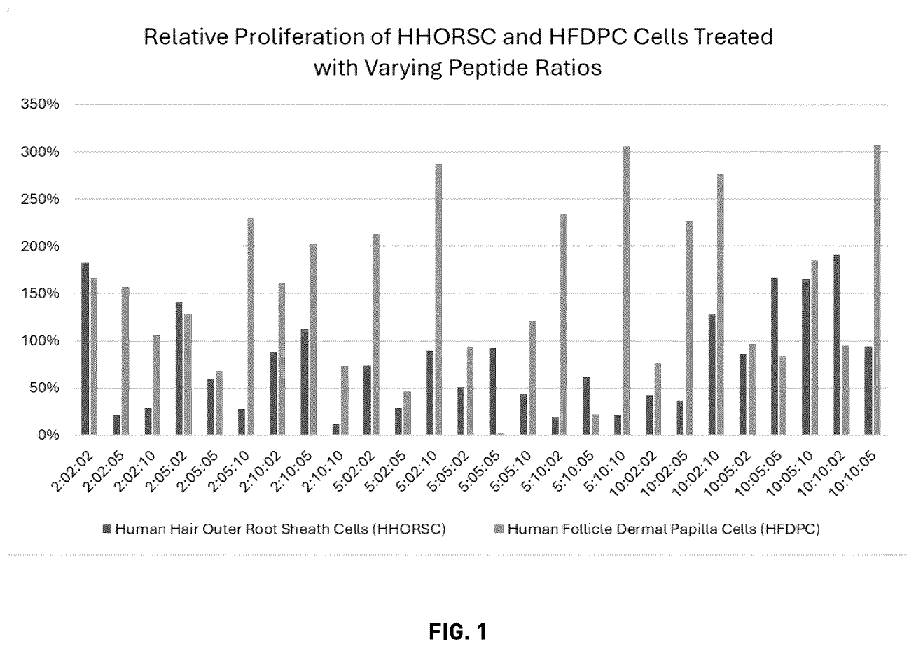

To determine the optimal treatment conditions, multiple peptide ratios of GHK-Cu, Thymosin Beta-4, and BPC-157 were evaluated for their effects on HHORSC and HFDPC proliferation using the MTT assay. The results demonstrated a dose-dependent response, with specific peptide ratios significantly enhancing metabolic activity and cell growth in both cell types (FIG. 1).

The most effective peptide ratio, 10:5:10, resulted in a 165% increase in proliferation for HHORSC and a 185% increase in HFDPC compared to untreated controls. This ratio was selected because it promoted strong proliferation in both cell lines rather than favoring one over the other, ensuring balanced efficacy across key follicular cell populations. Additionally, while proliferation levels were significantly increased, they remained within an optimal range, avoiding excessive cellular overgrowth that could indicate uncontrolled stimulation. These findings guided the selection of 10:5:10 as the optimized ratio for subsequent assays, evaluating its impact on wound healing, spheroid formation, and proteomic expression to further assess its therapeutic potential.

### Proteomic and Pathway Analysis of Peptide Treatment in Hair Follicle Cell Populations

Proteomic analysis revealed significant changes in key growth factors, inflammatory markers, and extracellular matrix regulators following treatment with the peptide combination in human epidermal keratinocytes (HEK), human hair outer root sheath cells (HHORSC), and human follicle dermal papilla cells (HFDPC). These findings, shown in Table 1 below, provide molecular-level validation of the peptide combination's ability to promote follicular health through enhanced proliferation, reduced inflammation, and extracellular matrix remodeling.

In HEK cells, notable increases were observed in VEGF (+11.76 pg/mL) and VEGF-D (+68.96 pg/mL), both of which play critical roles in vascularization and follicular nourishment. A moderate reduction in EGF (−9.08 pg/mL) and EGF receptor (−56.55 pg/mL) suggests a balanced modulation of epidermal proliferation, preventing excessive keratinocyte differentiation. Additionally, a strong downregulation of inflammatory markers such as IL-8 (−688.66 pg/mL) and MCP-1 (−377.96 pg/mL) suggests a significant reduction in follicular stress and immune infiltration, contributing to an improved microenvironment for follicular regeneration.

In HHORSC, key growth factors bFGF (+56.3 pg/mL) and FGF-7 (+10.9 pg/mL) showed substantial increases, directly supporting follicular proliferation and outer root sheath cell viability. IGF-I exhibited a robust increase (+232.25 pg/mL), underscoring its role in cellular growth and follicle maintenance. However, increases in TGF-β1 (+102.2 pg/mL) and TGF-β3 (+7.4 pg/mL) highlight the need for controlled activation, as excess levels in fibroblasts may contribute to scarring rather than regenerative benefits.

For HFDPC cells, proteomic shifts indicated a reduction in fibrosis-associated markers, with IGFBP-3 (−1801.8 pg/mL), IGFBP-6 (−1935.9 pg/mL), and TGF-β1 (−145.02 pg/mL) significantly downregulated, suggesting a decrease in excessive extracellular matrix deposition. Meanwhile, VEGF (+11.3 pg/mL) and EGF-R (+8.4 pg/mL) maintained levels conducive to hair follicle support without overstimulation.

These findings collectively demonstrate that the peptide combination modulates key biological pathways essential for hair follicle regeneration. The reduction of inflammatory mediators, combined with the enhancement of growth factors in a controlled manner, supports the therapeutic potential of this peptide treatment in mitigating follicular miniaturization and promoting sustained hair growth.

TABLE 1
Proteomic analysis of HEK, HHORSC, and HFDPC cells following treatment
with the peptide combination compared to untreated cells.

| | | | |
|---|---|---|---|
| | | Change | |
| Category | Factor | (pg/ml) | Benefit |

HEK (Human Epidermal Keratinocytes)

| | | | |
|---|---|---|---|
| Growth | VEGF | +11.76 | Enhances vascularization and follicular |
| Factors & | | | nourishment |
| Proliferation | VEGF-D | +68.96 | Improves angiogenesis and follicular |
| | | | nourishment |
| | EGF | −9.08 | Slight decrease may help balance epidermal |
| | | | differentiation |
| | EGF R | −56.55 | Receptor modulation prevents overactivation, |
| | | | which can suppress keratinocyte |
| | | | differentiation |
| Inflammation | IL-4 | +2.68 | Anti-inflammatory, supports regeneration |
| & Immune | IL-12p70 | +23.5 | Immune modulation for reducing excessive |
| Modulation | | | inflammation |
| | IL-1a | −9.39 | Reduces follicular stress |
| | IL-8 | −688.66 | Strong reduction; decreases follicular stress |
| | | | and immune infiltration |
| | MCP-1 | −377.96 | Large reduction; may reduce immune cell |
| | | | infiltration and follicular stress |
| | TNF-α | −7.78 | Reduces catagen phase induction, supporting |
| | | | hair retention |
| Fibrosis & | MMP-9 | −9.38 | Decreases matrix degradation and potential |
| ECM | | | fibrosis |
| Regulation | BMP-5 | −81.13 | Reduction may prevent excessive suppression |
| | | | of follicular activity |
| Neurotrophic | GDF-15 | +3.54 | Supports cellular stress response but should |
| & Other | | | be moderated |
| Signaling | | | |

HHORSC (Human Hair Outer Root Sheath Cells)

| | | | |
|---|---|---|---|
| Growth | bFGF | +56.3 | Supports follicular cell proliferation |
| Factors & | EGF R | +19.6 | Enhances epidermal cell proliferation and |
| Proliferation | | | follicle maintenance |
| | FGF-7 | +10.9 | Essential for outer root sheath cell |
| | | | proliferation |
| | IGF-I | +232.25 | Increase supports follicular proliferation; |
| | | | excess in fibroblasts can lead to fibrosis |
| | VEGF | +18.2 | Improves vascularization and blood supply to |
| | | | hair follicles |
| Inflammation | IL-1a | −3.3 | Reduces follicular stress |
| & Immune | IL-1b | −1.5 | Decreases inflammation-induced follicular |
| | | | damage |
| Modulation | IL-6 | +17.9 | Mild increase may support regeneration, but |
| | | | excess can be inflammatory |
| Fibrosis & | TGF-β1 | +102.2 | Supports follicular stem cell activation, but |
| ECM | | | excess in fibroblasts can lead to scarring |
| Regulation | TGF-β3 | +7.4 | Anti-fibrotic and supports follicular |
| | | | remodeling; excess in fibroblasts may |
| | | | contribute to scarring |

HFDPC (Human Follicle Dermal Papilla Cells)

| | | | |
|---|---|---|---|
| Growth | EGF R | +8.4 | Enhances epidermal cell proliferation and |
| Factors & | | | follicle maintenance |
| Proliferation | VEGF | +11.3 | Improves vascularization and blood supply to |
| | | | hair follicles |
| Fibrosis & | IGFBP-3 | −1801.8 | Reduction may increase bioavailable IGF-I, |
| ECM | | | promoting cellular proliferation |
| Regulation | IGFBP-6 | −1935.9 | Lower levels may enhance IGF-I activity, |
| | | | supporting cell proliferation |
| | HGF | −410.7 | Lower levels may reduce excessive fibroblast |
| | | | activation while maintaining ECM |
| | | | remodeling |
| | TGF-β1 | −145.02 | Lower levels may reduce fibrosis and |
| | | | excessive ECM deposition |
| | IGF-I | −230.1 | Lower levels may help reduce fibrosis, |
| | | | balancing its proliferative role in HHORSC |
| | TGF-β3 | −9.36 | Reduction in fibroblasts may help prevent |
| | | | excessive scarring |
| Neurotrophic | GDNF | −3.41 | May regulate nerve-related effects on |
| & Other | | | follicular signaling |
| Signaling | | | |

### Wound Healing Assay

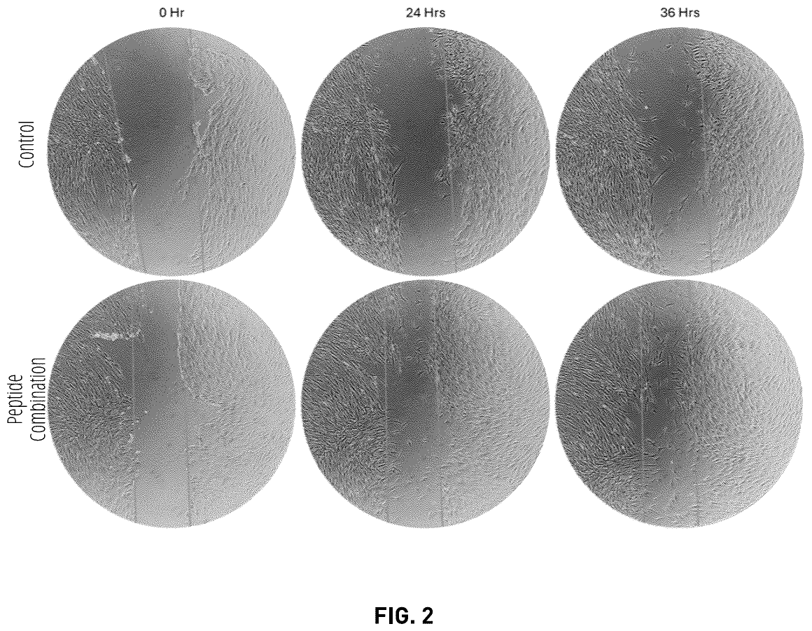

Treatment with the peptide combination significantly enhanced cellular migration in HHORSC cells, a key process in follicular regeneration and wound repair (FIG. 2). By 36 hours, peptide-treated wells exhibited 53.3% wound closure, compared to 39.4% in the control, representing a 13.9% greater closure rate with peptide treatment. This increased migration suggests that the peptide combination actively promotes the movement of follicular cells, which is essential for maintaining hair follicle health and accelerating tissue recovery.

### Spheroid Formation Assay

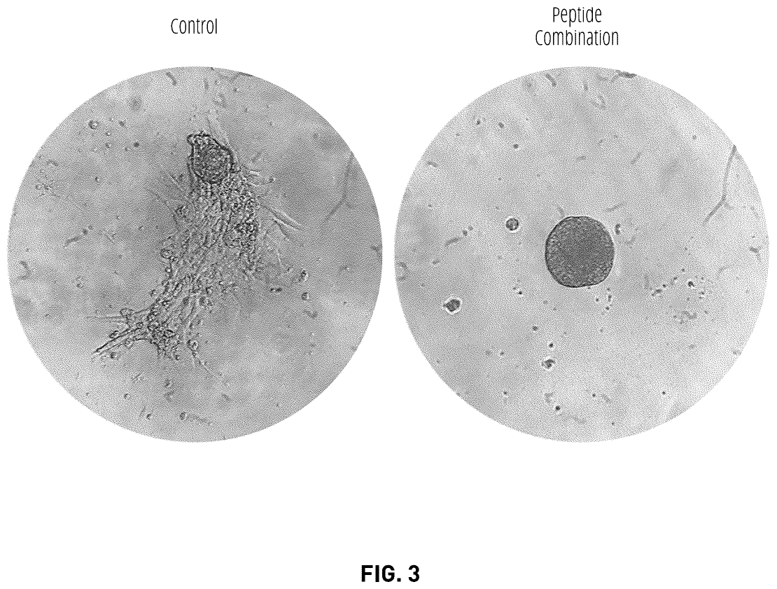

Treatment with the peptide combination resulted in the formation of more compact and structurally cohesive spheroids in HHORSC cells, indicating enhanced cell-cell adhesion and improved cellular viability (FIG. 3). The increased spheroid tightness suggests a more favorable microenvironment for maintaining outer root sheath cell integrity, a critical factor in follicular regeneration and hair growth. HHORSC cells were selected for this assay due to their essential role in providing structural support to the hair follicle and facilitating interactions between dermal papilla cells and the surrounding epithelial layers. Improved spheroid formation in this cell type reinforces the potential of the peptide combination in strengthening follicular architecture and supporting sustained hair growth.

### Discussion

### Molecular Mechanisms Identified Through Proteomics

Proteomic analysis revealed that treatment with the peptide combination led to significant changes in key growth factors, inflammatory markers, and extracellular matrix regulators across multiple follicular cell populations. In HHORSC cells, levels of bFGF, IGF-I, and VEGF were notably increased, supporting follicular proliferation, angiogenesis, and tissue repair. Additionally, the downregulation of inflammatory mediators such as IL-8, MCP-1, and TNF-α in HEK cells suggests a reduction in immune-driven follicular stress, which is critical for sustaining the hair growth cycle. The reduction of fibrosis-associated proteins, including IGFBP-3, IGFBP-6, and TGF-β1 in HFDPC cells, further indicates that the peptide combination may help maintain follicular integrity by preventing excessive extracellular matrix deposition. These combined proteomic shifts highlight the peptide combination's ability to balance regenerative pathways while mitigating inflammatory and fibrotic responses that contribute to follicular miniaturization.

### Functional Validation of Proteomic Findings

The enhanced expression of growth factors was reflected in the MTT proliferation assay, where the optimized 10:5:10 peptide ratio led to a 165% increase in HHORSC proliferation and a 185% increase in HFDPC proliferation. These findings confirm that the peptide combination supports robust follicular cell expansion while maintaining controlled proliferation, ensuring its efficacy without overstimulation. The observed balance in proliferative effects across both cell types further reinforces the peptide combination's role in creating a favorable environment for sustained hair growth.

The wound healing assay provided additional functional validation, showing a 13.9% greater closure rate in HHORSC cells treated with the peptide combination compared to controls. This aligns with proteomic findings of increased bFGF and VEGF expression, both of which are known to enhance cellular migration and tissue repair. The accelerated closure suggests that the peptide combination not only promotes cell proliferation but also facilitates the movement of follicular cells, which is essential for maintaining follicular health and promoting hair regrowth.

The spheroid formation assay demonstrated that peptide treatment resulted in tighter and more cohesive spheroids in HHORSC cells, indicating improved cell-cell adhesion and follicular structural integrity. This suggests that the peptide combination not only enhances individual cell function but also promotes a supportive microenvironment for sustained follicular health. The ability of HHORSC cells to form structurally stable spheroids under peptide treatment further supports the hypothesis that the peptide combination strengthens follicular architecture, reinforcing its potential for long-term hair follicle maintenance.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “substantially” and “about.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. All ranges also include both endpoints.

Similarly, in the above description of embodiments, various features are sometimes grouped together in a single embodiment or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A composition comprising:
a peptide combination including at least two peptides selected from:
a first peptide having at least 80% identity to SEQ ID NO: 1;
a second peptide having at least 80% identity to SEQ ID NO: 2; and
a glycyl-L-histidyl-L-lysine-copper complex (GHK-Cu).

2. (canceled)

3. The composition of claim 1, wherein the first peptide is thymosin beta-4.

4. (canceled)

5. The composition of claim 1, wherein the second peptide is BPC-157.

6. (canceled)

7. The composition of claim 1, wherein the peptide combination comprises each of the first peptide, the second peptide, and GHK-Cu.

8. The composition of claim 7, wherein the peptides are present in approximately equal amounts.

9. The composition of claim 7, wherein GHK-Cu and the second peptide are each present at a weight percentage ratio of about 1:1 to about 3:1 with respect to the first peptide.

10-18. (canceled)

19. The composition of claim 1, wherein the peptide combination includes a peptide complex in which two of the at least two peptides are linked by a crosslinking agent.

20. (canceled)

21. (canceled)

22. The composition of claim 19, comprising each of GHK-Cu, the first peptide, and the second peptide; and
further comprising a second crosslinking agent,
wherein the crosslinking agent links an N-terminus of GHK-Cu with a first lysine residue of the first peptide, and the second crosslinking agent links a second lysine of the first peptide to a C-terminus of the second peptide.

23-50. (canceled)

51. A topical formulation for treating hair loss, comprising:
a vehicle formulated for topical application, and comprising a peptide combination including at least two peptides selected from:
a first peptide having at least 80% identity to SEQ ID NO: 1;
a second peptide having at least 80% identity to SEQ ID NO: 2; and
glycyl-L-histidyl-L-lysine-copper complex (GHK-Cu).

52. (canceled)

53. The topical formulation of claim 51, wherein the first peptide is thymosin beta-4.

54. (canceled)

55. The topical formulation of claim 51, wherein the second peptide is BPC-157.

56. The topical formulation of claim 51, wherein the peptide combination comprises each of the first peptide, the second peptide, and GHK-Cu.

57. The topical formulation of claim 56, wherein GHK-Cu and the second peptide are present at a weight percentage ratio of about 1:1 to about 3:1 with respect to the first peptide.

58. (canceled)

59. The topical formulation of claim 51, wherein the vehicle is a solvent in which the peptide combination is dissolved.

60. The topical formulation of claim 51, wherein the vehicle is a shampoo, a dry shampoo, a rinse-off conditioner, a leave-in conditioner, a conditioning mask, a hair oil, or a scalp serum.

61-69. (canceled)

70. A method of treating hair loss in a subject, comprising:
applying an effective amount of the topical formulation of claim 60 to an area of skin of the subject that is affected by hair loss.

71. The method of claim 70, wherein the vehicle is a shampoo, rinse-off conditioner, leave-in conditioner or conditioning mask, further comprising wetting the area before applying.

72. The method of claim 71, wherein the vehicle is a shampoo, rinse-off conditioner or conditioning mask, further comprising rinsing the topical formulation from the area with water after a period of time.

73. The composition of claim 1, further comprising a solvent in which the peptide combination is dissolved.

74. The composition of claim 1, wherein the composition is a powder having a moisture content of no more than about 5 wt %.


SEQUENCE LISTING (2 sequences)

SEQ ID NO: 1 - protein, 43 aa, Homo sapiens
SDKPDMAEIEKFDKSKLKKTETQEKNPLPSKETIEQEKQAGES

SEQ ID NO: 2 - protein, 15 aa, Homo sapiens
GEPPPGKPADDAGLV